(19) United States
(12) Patent Application Publication
(10) Pub. No.: US 2009/0130757 A1
(43) Pub. Date: May 21, 2009

(54) BIOREACTOR WITH MIXER AND SPARGER

(76) Inventor: Alexandre N. Terentiev, Lexington, KY (US)

Correspondence Address: KING & SCHICKLI, PLLC, 247 NORTH BROADWAY, LEXINGTON, KY, 40507, US

(21) Appl. No.: 11/913,077

(22) PCT Filed: Oct. 26, 2006
(86) PCT No.: PCT/US06/42161

§ 371 Date: Oct. 30, 2007

Related U.S. Application Data
(60) Provisional application No. 60/730,489, filed on Oct. 26, 2005.
(60) Provisional application No. 60/841,012, filed on Aug. 30, 2006.

(51) Int. Cl. C12N 5/06; C12M 1/00

(52) U.S. Cl. 435/394; 435/295.1

(57) ABSTRACT

A bioreactor and related methods are for use in bioprocessing in which a fluid is received and agitated using an internal fluid-agitating element driven by an external motive device. In one embodiment, the bioreactor includes a mixer and a movable sparger. The mixer may take the form of a rotational wand mixer, and may further comprise a magnetic impeller. In another embodiment, the bioreactor includes a rotational wand mixer and an integral sparger. In still another embodiment, the bioreactor comprises a bag including a rotational wand mixer having rigid blades attached thereto.

This application claims the benefit of U.S. Provisional Patent Application Ser. No. 60/730,489, filed Oct. 26, 2005 and U.S. Provisional Patent Application Ser. No. 60/841,012, filed Aug. 30, 2006, the disclosures of which are incorporated herein by reference.

### TECHNICAL FIELD

The present invention relates generally to vessels for holding fluids and, more particularly, to a bioreactor including a mixer and a sparger.

### BACKGROUND OF THE INVENTION

Typically, a bioreactor comprises a sterile vessel designed to provide optimum growth conditions for a cell culture. To create such optimum conditions, the cell culture in the bioreactor often needs to be mixed during cell growth. Also, gases like oxygen need to be delivered to cell culture to maintain proper conditions for cell metabolism. pH and dissolved oxygen probes are normally used to control and maintain parameters at the optimal levels.

Usually, mixing impellers uniformly disperse gas, such as in the form of bubbles, throughout the volume of bioreactor. The gas bubbles may be formed by introducing pressurized gas to the fluid through a sparger or sparging element, which usually has small holes or pores that break the gas incoming gas into fine bubbles. Since small bubbles have a large surface to volume ratio, diffusion of the gas into fluid is greatly accelerated.

Traditional bioreactors comprise vessels made of stainless steel or glass. However, the current trend in biopharmaceutical manufacturing is to switch from such vessels to more readily disposable ones and, in particular, flexible plastic bags. When sterilized, disposable bags eliminate time consuming cleaning and validation, resulting in reduced cross contamination risk. Storage and transportation costs are also reduced.

A mixing bag including a sleeve and rotational rigid rod introduced inside the sleeve is described in commonly assigned U.S. Pat. No. 6,494,613. The rigid rod introduced into the tube or sleeve is rotated by an external motor to create mixing action inside the bag. However, mixing alone is insufficient to operate the bag in bioreactor regime.

Accordingly, a need is identified for an improved bioreactor for use in bioprocessing applications. The bioreactor would be easy to construct in an expensive fashion, and would be readily disposable and otherwise simple to use. Despite its simplicity, the apparatus provided would result in an unprecedented level of mixing ability, including possibly under sterile conditions, while at the same time facilitating cell growth by ensuring the full distribution of bubbles from any sparger provided throughout the fluid to improve the concentration of dissolved gas.

### SUMMARY OF THE INVENTION

One aspect of the invention is an apparatus intended for use in bioprocessing with a fluid. The apparatus comprises a vessel having an interior compartment capable of holding the fluid. A mixer rotatable about an axis of rotation agitates the fluid, and a sparger generates bubbles within the fluid. A motive device is also provided for moving the sparger about the axis of rotation. The mixing action in the fluid created by the mixer combined with the movement of the sparger about the axis of rotation helps distribute the bubbles from the sparger throughout the fluid to improve the concentration of dissolved gas.

In one embodiment, the mixer couples to the sparger. In another embodiment, the motive device comprises a motor for rotating the sparger about the axis of rotation. In still another embodiment, the motive device rotates the mixer.

In a particularly preferred alternative embodiment, the vessel comprises a bag including a flexible sleeve projecting within the interior compartment. Furthermore, the mixer comprises a mixing rod for insertion in the sleeve. The mixing rod induces rotation in the sleeve to agitate the fluid without exposing the mixing rod to the fluid. In any of these embodiments, the sparger may comprise an elongated flexible tube for delivering the gas to the fluid in the vessel by way of a porous material, and the mixer may include rigid blades.

In accordance with another aspect of the invention, a bioreactor intended for receiving a fluid is disclosed. The bioreactor comprises a vessel having an interior compartment capable of receiving and holding the fluid. The vessel includes an elongated flexible sleeve projecting within the interior compartment and coupled to the vessel by way of a static seal. A mixer is provided for insertion in the sleeve that induces rotation in the sleeve to agitate the fluid without exposing the mixer to the fluid without causing the sleeve to bodily rotate about a longitudinal axis. A sparger is also provided for forming bubbles from a gas supplied to the fluid in the vessel. As a result of this arrangement, the mixing action in the fluid created by the mixer helps distribute the bubbles from the sparger throughout the fluid to improve the concentration of dissolved gas.

Preferably, a weld connects the sparger to the vessel to form the static seal, and the sparger lies adjacent a floor of the vessel. Most preferably, the sparger couples to the flexible sleeve, and comprises an elongated flexible tube for delivering the gas to the fluid in the vessel by way of a porous material. The vessel also preferably includes at least one port in communication with the elongated flexible tube, which may be external to the interior compartment.

In one embodiment, the bioreactor further comprises a second mixer for agitating the fluid. Preferably, the second mixer comprises an impeller adjacent a floor of the vessel. To enhance the mixing action provided, the sleeve may carry rigid blades. Furthermore, the vessel may comprise a flexible bag to promote disposability.

In accordance with another aspect of the invention, a bioreactor intended for receiving a fluid useful in culturing cells is disclosed. The bioreactor comprises a vessel having an interior compartment capable of receiving and holding the fluid, as well as a flexible sleeve extending into the interior compartment that may carry rigid blades. A mixer positioned in the sleeve induces rotation therein to agitate the fluid without the mixer contacting the fluid. A sparger is coupled to the mixer for forming bubbles from a gas supplied to the fluid in the bag, such as by way of an external port communicating with a flexible tube or like conduit. As in the other embodiments, the mixing action in the fluid created by the mixer helps distribute the bubbles from the sparger coupled thereto throughout the fluid to improve the concentration of dissolved gas.

In accordance with a further aspect of the invention, an apparatus intended for receiving and agitating a fluid is disclosed. The apparatus comprises a vessel, such as a flexible bag, having an interior compartment capable of receiving and holding the fluid. First and second spaced, independently movable fluid-agitating elements agitate the fluid in the vessel or bag.

In one embodiment, first and second motors independently rotate the fluid-agitating elements. Preferably, at least one of the first and second fluid-agitating elements comprises an impeller positioned within an interior compartment of the vessel and rotated by way of a magnetic coupling. Alternatively, at least one of the first and second fluid-agitating elements comprises a magnetic impeller levitated by a superconducting element. Still another alternative is to provide a sleeve projecting into the interior compartment of the vessel, in which case the fluid-agitating element comprises a rigid rod positioned in the sleeve and rotated by a motor. In any case, it is preferable that the first and second fluid-agitating elements are opposed.

In accordance with yet a further aspect of the invention, a method of bioprocessing using a fluid held in a vessel is disclosed. The method comprises rotating a mixer in the vessel to agitate the fluid while simultaneously moving (e.g., rotating) a sparger about the vessel to help distribute gas throughout the fluid and improve the concentration of dissolved gas. The method preferably further comprises the step of coupling the mixer and sparger, as well as the additional steps of providing a sleeve within an interior compartment of the vessel and inserting a mixing rod in the sleeve.

Yet another aspect of the invention is a method of manufacturing a bioreactor. The method comprises providing a vessel having an interior compartment capable of receiving and holding a fluid. An elongated flexible sleeve projects into the interior compartment and couples to the vessel by way of a static seal. The method further comprises attaching to the vessel a sparger for forming bubbles from a gas supplied to the fluid. Still further, the method comprises moving the sleeve within the interior compartment without rotating the sleeve about the longitudinal axis to distribute the bubbles throughout the fluid.

In one embodiment, the attaching step comprises attaching the sleeve to the sparger, such that the moving step includes moving the sparger. In another, the attaching step comprises welding the sparger to the vessel. In still another, the attaching step comprises connecting a flexible tube forming part of the sparger to a port associated with the vessel.

A further aspect of the invention is a method of agitating a fluid comprising providing a vessel in the form of a flexible bag and providing first and second spaced, independently movable fluid-agitating elements for agitating the fluid in the bag. The method may further include the step of providing first and second motors for independently rotating the fluid-agitating elements. In the case where at least one of the first and second fluid-agitating elements comprises an impeller positioned within an interior compartment of the vessel, the method comprises rotating the impeller by way of a magnetic coupling. In the alternate case where a sleeve projects into the interior compartment of the vessel, and the fluid-agitating element comprises a rigid rod positioned in the sleeve. The method then further includes the step of using the rigid rod to induce rotation of the sleeve within the interior compartment.

### BRIEF DESCRIPTION OF THE DRAWINGS

FIG. 1 is a partially cutaway perspective view of a bioreactor vessel according to one embodiment of the invention;

FIG. 2 is a partially cutaway perspective view of a bioreactor vessel according to another embodiment of the invention;

FIG. 2a is a partially cutaway, enlarged, cross-sectional view of the vessel of FIG. 2;

FIG. 3 is a partially cutaway perspective view of a bioreactor vessel according to yet another embodiment of the invention;

FIG. 3a is a partially cutaway, enlarged, cross-sectional view of the vessel of FIG. 3; and

FIG. 4 is a partially cutaway perspective view of a bioreactor vessel according to yet another embodiment of the invention.

### DETAILED DESCRIPTION OF THE INVENTION

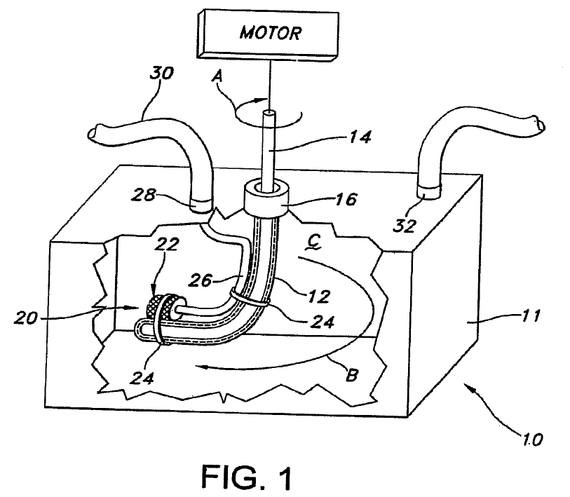

Reference is now made to FIG. 1, which discloses one embodiment of the vessel 10 of the present invention for use as a bioreactor. The vessel 10 in this embodiment comprises a collapsible bag 11 including an inner compartment C for receiving and holding a fluid (which term incorporates liquids, gases, solid suspensions, and the like) for being agitated or mixed. As described in the above-referenced '613 patent, in the case of a collapsible bag 11, support may be provided by an outer rigid container (not shown) so as to provide support for the fluid as well. However, it is within the broadest aspects of the invention for the vessel 10 to itself comprise a rigid container made of any suitable material, including rigid plastic, glass, metal, or the like.

In the embodiment shown in FIG. 1, a flexible sleeve 12 projects in the interior compartment of the vessel 10 or bag 11 for agitating any fluid present. The sleeve 12 includes a closed end in the compartment C so as to form a cavity within the vessel 10 or bag 11 and an open end for receiving a mixer, such as a rigid mixing rod 14 having at least some degree of curvature. Adjacent the open end, the sleeve 12 is coupled to the vessel 10 or bag 11 and projects therein by way of a seal 16. This seal 16 is preferably hermetic, and may be statically formed by a circumferential weld formed between the open end of the sleeve 12 and an opening or hole in the vessel 10 or bag 11 so as to give it an annular shape. As a result of this type of attachment, the sleeve 12 is incapable of bodily rotating about its own longitudinal axis, but can still move about the interior compartment C in order to agitate the fluid and provide the desired mixing action (note action arrow B).

The distal end of the mixing rod 14 may be inserted into the sleeve 12 so as to engage the distal end thereof, and may be supported by optional spacers as disclosed in the '613 patent. The opposite end of the rod 14 may in turn couple with or connect to a motive device, such as a rotational motor. Upon being actuated, this motor bodily rotates the rod 14 about its own longitudinal axis (note action arrow A). As a result of this movement, the sleeve 12 rotates about the interior compartment C to agitate the fluid, but without rotating about its own longitudinal axis. Moreover, by virtue of the sleeve 12 being closed within the interior compartment C, the mixing rod 14 never contacts the fluid directly, and can thus be withdrawn and reused in a different mixing application with this or a similar type of vessel 10 without being cleaned.

In accordance with one aspect of the invention, the bioreactor vessel 10 includes a movable sparger 20. In the embodiment of FIG. 1, the sparger 20 comprises a porous material 22 connected to the distal end of the sleeve 12. The connection may be by way of a band 24, tie, fastener or like coupling means, or instead may be by way of welding or a like type of connection (e.g., an adhesive).

An internal conduit, which may be a rigid pipe but is shown as comprising a flexible tube 26, in turn connects the porous material 22 to a port 28 formed in the vessel 10 or bag 11. This tube 26 may also be coupled to the sleeve 12, such as by using another band 24, tie, or like coupler. The port 28, which may be hermetically sealed, couples with an external supply line, such as conduit 30, for supplying gas from a remote source (not shown) to the tube 26 and hence the porous material 22. However, it is within the broadest aspects of the invention to use any opening formed in the vessel 10 or bag 11 as the port 28 for receiving the flexible tube 26, which may in such case simply be coextensive with the supply line 30.

The porous material 22 is preferably such that it allows for the “one way” passage of fluid only; in other words, gas can pass through the material into an adjacent fluid, but the fluid and gas cannot pass through the porous material into the tube 26. Consequently, in the case where the vessel 10 or bag 11 is hermetically sealed, a vent or exhaust port 32 may also be provided. Preferably, a filter (not shown) or like means is associated with the port 32 for preventing the introduction of undesired contaminants, such as microorganisms or the like.

In operation, the rod 14 of the mixer is rotated to induce movement in the sleeve 12, which again does not rotate about its own longitudinal axis. This movement causes the porous material 22 of the sparger 20 to move simultaneously with the sleeve 12 about the interior compartment C and preferably adjacent the floor of the vessel 10. This movement helps to distribute the bubbles from the sparger 20 throughout the interior compartment C and thereby improves the concentration of dissolved gas in the fluid (which may be controlled by simply adjusting the supply of gas to the supply tube 30).

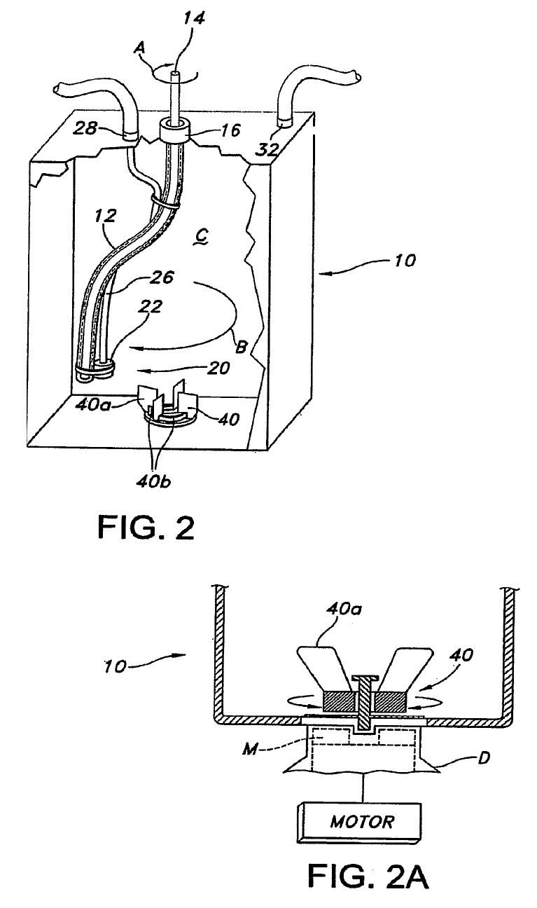

FIG. 2 illustrates an alternative embodiment, which in many respects is similar to the one described in FIG. 1. However, in addition to the agitation of the fluid created by the “wand” mixer, additional agitating capacity is provided by a second mixer positioned in the vessel 10. In the embodiment of FIG. 2, this second mixer preferably comprises a rotatable stirrer and, most preferably, a bodily rotatable impeller 40, such as the one shown having one or more rigid blades 40a. Preferably, this impeller 40 lies adjacent a floor of the vessel 10, which again may be rigid or a flexible bag. The most preferred positioning is generally opposite the “wand” mixer in the illustrated embodiment, which thereby ensures that the agitation is provided in both the upper and lower portions of the internal compartment C.

While it is within the broadest aspects the invention for the second mixer to comprise a second wand mixer (not shown), the preferred use of a bodily rotatable impeller 40 may further comprise one or more magnets 40b, either alone or in combination with a matrix material. These magnets 40b may connect through the vessel 10 with an external motive device (note device D and drive magnet M) by way of a magnetic coupling, which coupling may then be used to induce rotation for agitating the fluid. The vessel 10 may further include a post P for receiving the impeller 40, and preferably a bearing providing support for it at least in a resting position. This bearing may comprise, for example, the peripheral seating surface shown as part of the rigid disc-shaped structure supporting the post P, or a separate roller bearing element supporting impeller 40. The impeller 40 may also be free of direct connection to the post P and levitated and/or rotated by a thermally shielded superconducting element in place of or in addition to the drive magnet M. In any case, this embodiment can be characterized as a collapsible, hermetically sealed vessel 10 having an interior compartment capable of receiving and holding the fluid, with first and second independently rotatable fluid-agitating elements for thoroughly mixing the fluid and/or enhancing the distribution of the bubbles from the sparger 20 throughout the fluid to improve the concentration of dissolved gas.

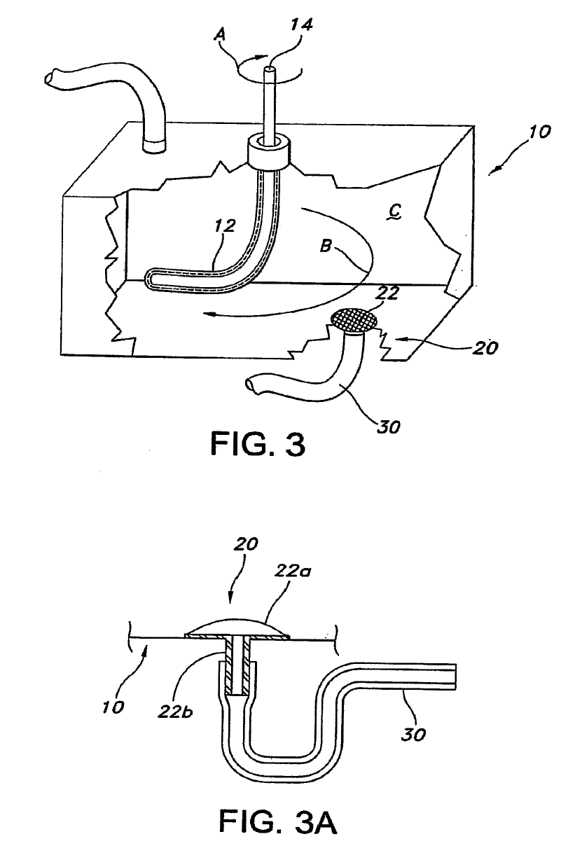

FIG. 3 illustrates yet another embodiment in which a rotatable sleeve 12 is provided in a mixing vessel 10. Unlike the embodiment of FIGS. 1 and 2, gas diffusion in this embodiment is provided by a sparger 20 integrally formed with the vessel 10, such as by being connected to the floor thereof. The sparger 20 comprises a porous material 22 connected to the external conduit 30 for delivering gas from an external supply. As in the other embodiments, the porous material 22 may comprise a gas permeable or perforated film 22a for introducing gaseous sparging bubbles into the interior compartment C of the vessel 10 (see FIG. 3a). The film 22a may be connected directly to the vessel 10, such as by welding, or may be connected to a rigid port 22b for coupling with the conduit 30.

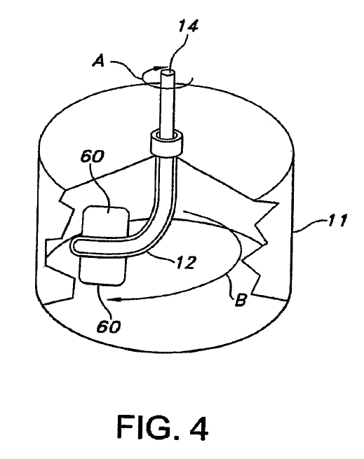

FIG. 4 illustrates another embodiment of a vessel 10 including a rotational wand mixer similar in basic construction to the one shown in FIG. 1. However, in this embodiment, at least one and preferably a pair of opposed rigid blades 60 attach directly to the flexible sleeve 12, such as by using a weld, fastener, or like means of connection. Consequently, these blades 60 move about the fluid and help to enhance agitation as the sleeve 12 rotates about the interior compartment of the vessel 10 (such as the bag 11, which is shown as being cylindrical for purposes of illustration only), but without rotating about its own longitudinal axis (and thereby avoiding the need for a dynamic seal). Preferably, the blades 60 extend radially and, most preferably, in an opposed fashion, and may be spaced along the longitudinal axis of the sleeve 12. Sparging function may be provided by an optional sparger (not shown in FIG. 4), which may be coupled to the sleeve 12 (as in FIGS. 1 and 2) or integral with the vessel 10 (as in FIG. 3).

Obvious modifications or variations are possible in light of the above teachings. For example, it may also be desirable to provide disposable means in the vessel 10 to facilitate sensing characteristics of the fluid, such as the pH, oxygen content, temperature, etc. As briefly noted above, the porous material 22 could also be integrally formed with the sleeve 12, as could the rigid blades 60. The foregoing descriptions of various embodiments of the present inventions have been presented for purposes of illustration and description. These descriptions are not intended to be exhaustive or to limit the invention to the precise forms disclosed. The embodiments described provide the best illustration of the principles of the invention and its practical applications to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

1. An apparatus intended for use in bioprocessing with a fluid, comprising:
a vessel having an interior compartment capable of holding the fluid;
a mixer rotatable about an axis of rotation to agitate the fluid;
a sparger for supplying a gas to the fluid; and
a motive device for moving the sparger about the axis of rotation,
whereby the mixing action in the fluid created by the mixer combined with the movement of the sparger about the axis of rotation helps distribute the sparger throughout the fluid to improve the concentration of dissolved gas.

2-4. (canceled)

5. The apparatus of claim 1, wherein the vessel comprises a bag including a flexible sleeve projecting within the interior compartment.

6. The apparatus of claim 5, wherein the mixer comprises a mixing rod for insertion in the sleeve that induces rotation in the sleeve to agitate the fluid without exposing the mixing rod to the fluid.

7. The apparatus of claim 6, wherein the sparger comprises an elongated flexible tube for delivering the gas to the fluid in the vessel by way of a porous material.

8. (canceled)

9. A bioreactor intended for receiving a fluid, comprising:
a vessel having an interior compartment capable of receiving and holding the fluid, the vessel including an elongated flexible sleeve projecting within the interior compartment and coupled to the vessel by way of a static seal;
a mixer for insertion in the sleeve that induces rotation in the sleeve to agitate the fluid without exposing the mixer to the fluid without causing the sleeve to rotate about a longitudinal axis; and
a sparger for supplying a gas to the fluid in the vessel,
whereby the mixing action in the fluid created by the mixer helps distribute the sparger throughout the fluid to improve the concentration of dissolved gas.

10-15. (canceled)

16. The bioreactor of claim 9, further including a second mixer for agitating the fluid.

17. The bioreactor of claim 16, wherein the second mixer comprises an impeller.

18-19. (canceled)

20. The bioreactor of claim 9, wherein the vessel comprises a flexible bag.

21. A bioreactor intended for receiving a fluid useful in culturing cells, comprising:
a vessel having an interior compartment capable of receiving and holding the fluid, the vessel including a flexible sleeve extending into the interior compartment;
a mixer positioned in the sleeve that induces rotation in the sleeve to agitate the fluid without the mixer contacting the fluid; and
a sparger coupled to the mixer for supplying a gas to the fluid in the bag,
whereby the mixing action in the fluid created by the mixer helps distribute the bubbles from the sparger throughout the fluid to improve the concentration of dissolved gas.

22. The bioreactor of claim 21, wherein the vessel comprises a flexible bag.

23. The bioreactor of claim 21, wherein the sparger includes a tube in communication at one end with a port in the vessel and at the other end with a porous material for forming the bubbles from the gas when supplied through the flexible tube.

24. The bioreactor of claim 21, wherein the vessel includes a vent port.

25. The bioreactor of claim 21, further including an impeller in the vessel.

26. The bioreactor of claim 21, further including rigid blades carried by the sleeve.

27. An apparatus intended for receiving and agitating a fluid, comprising:
a vessel having an interior compartment capable of receiving and holding the fluid; and
first and second spaced, independently movable fluid-agitating elements for agitating the fluid in the bag.

28-30. (canceled)

31. The apparatus of claim 27, further including a sleeve projecting into the interior compartment of the vessel, and wherein the fluid-agitating element comprises a rigid rod positioned in the sleeve.

32. The apparatus of claim 31, further including a motor for rotating the rigid rod.

33. (canceled)

34. The apparatus of claim 27, wherein the vessel comprises a flexible bag.

35. The apparatus of claim 27, further including a sparger.

36. A method of bioprocessing using a fluid held in a vessel, comprising:
rotating a mixer in the vessel to agitate the fluid; and
simultaneously moving a sparger about the vessel to help distribute the sparger throughout the fluid and improve the concentration of dissolved gas.

37-39. (canceled)

40. A method of manufacturing a bioreactor, comprising:
providing a vessel having an interior compartment capable of receiving and holding a fluid and including an elongated flexible sleeve projecting into the interior compartment and coupled to the vessel by way of a static seal;
attaching to the vessel a sparger for forming bubbles from a gas supplied to the fluid; and
moving the sleeve within the interior compartment without rotating the sleeve about the longitudinal axis to distribute the bubbles throughout the fluid.

41-44. (canceled)

45. A method of manufacturing a vessel for use in bioprocessing with a fluid, comprising:
providing a vessel for holding the fluid; and
providing first and second spaced, independently movable fluid-agitating elements for agitating the fluid in the vessel.

46-47. (canceled)

48. The method of claim 45, further including a sleeve projecting into the interior compartment of the vessel, and wherein the fluid-agitating element comprises a rigid rod positioned in the sleeve, and further including the step of using the rigid rod to induce rotation of the sleeve within the interior compartment.

49. The method of claim 45, further comprising the step of collapsing the vessel.

50. The method of claim 45, further comprising the step of sparging the fluid.
